(19) United States
(12) Patent Application Publication
(10) Pub. No.: US 2012/0120112 A1
(43) Pub. Date: May 17, 2012

(54) ELECTRICAL DEVICE, DISPLAY METHOD FOR ELECTRICAL DEVICE AND ELECTRICAL DEVICE READABLE STORAGE MEDIUM FOR STORING THEREOF

(75) Inventors: Ya-Ting TSAI, Taipei County (TW); Yun-Jung HSIEH, Chiayi City (TW)

(73) Assignee: INSTITUTE FOR INFORMATION INDUSTRY, TAIPEI (TW)

(21) Appl. No.: 12/964,726

(22) Filed: Dec. 9, 2010

(30) Foreign Application Priority Data
Nov. 15, 2010 (TW) 099139226

(51) Int. Cl. G09G 5/00; A61B 3/032

(52) U.S. Cl. 345/661; 351/239; 351/246

(57) ABSTRACT

An electrical device includes a display unit and a processing unit, which are electrically connected with each other. The processing unit includes an information receiving module, a weight obtaining module and a size adjusting module. The information receiving module receives physical information of a user. The weight obtaining module obtains a display weight according to the physical information of the user. The size adjusting module adjusts size of at least one display icon according to the display weight.

### RELATED APPLICATIONS

This application claims priority to Taiwan Application Serial Number 099139226, filed Nov. 15, 2010, which is herein incorporated by reference.

### BACKGROUND

1. Technical Field

The present invention relates to an electrical device, a display method for an electrical device and an electrical device readable storage medium with a computer program to execute a display method for an electrical device.

2. Description of Related Art

As 3C (Computer, Communications and Consumer) develops, many people utilize portable electrical devices in their daily life. Wherein, the most popular portable electrical devices include Personal Digital Assistants, mobile phones, smart phones etc.

Besides, more and more elder people have demand for portable electrical devices. Since portable electrical devices are designed to carry on, small display units are utilized for most portable electrical devices, which means that small display area can be utilized for display. However, it's hard for the elders to see the icons displayed on the small display unit. In addition, icons displayed on the portable devices can be set to bigger size, but the setting method is complicated and not convenient to the elders.

### SUMMARY

According to one embodiment of this invention, an electrical device is provided. The electrical device adjusts size of at least one display icon displayed on the display unit of the electrical device according to physical information of a user. The electrical device includes a display unit and a processing unit, which are electrically connected with each other. The processing unit includes an information receiving module, a weight obtaining module and a size adjusting module. The information receiving module receives physical information of a user. The weight obtaining module obtains a display size weight according to the physical information of the user. The size adjusting module adjusts size of at least one display icon displayed on the display unit according to the display size weight.

According to another embodiment of this invention, a display method for an electrical device is provided. In the display method, size of at least one display icon displayed on the display unit of the electrical device is adjusted according to physical information of a user. The display method may take the form of a computer program product stored on an electrical device readable storage medium having electrical device readable instructions embodied in the medium. The display method includes the following steps: physical information of a user is received. A display size weight is obtained according to the physical information of the user. Size of at least one display icon displayed on a display unit of the electrical device is adjusted according to the display size weight.

These and other features, aspects, and advantages of the present invention will become better understood with reference to the following description and appended claims. It is to be understood that both the foregoing general description and the following detailed description are by examples, and are intended to provide further explanation of the invention as claimed.

### BRIEF DESCRIPTION OF THE DRAWINGS

The invention can be more fully understood by reading the following detailed description of the embodiments, with reference made to the accompanying drawings as follows:

FIG. 1 illustrates a block diagram of an electrical device according to one embodiment of this invention;

FIG. 2 is an embodiment of a display appearance of the display unit 110 in FIG. 1; and

FIG. 3 is a flow diagram of a display method for an electrical device according to another embodiment of this invention.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

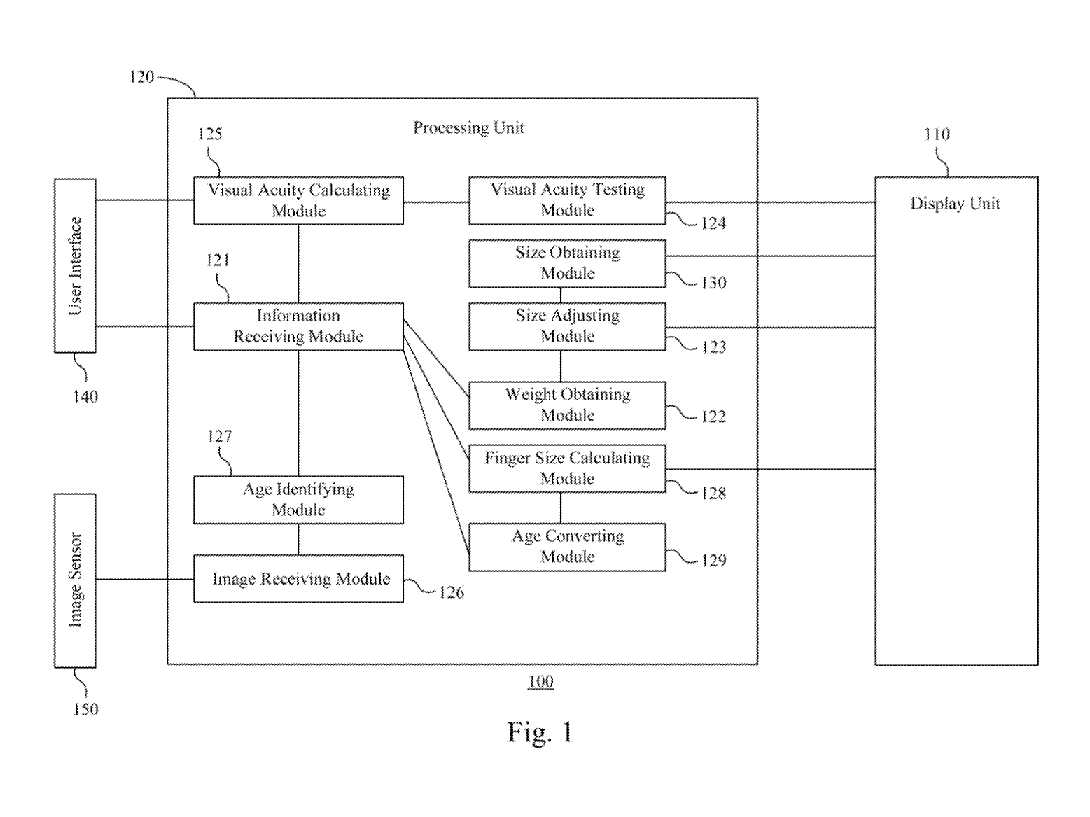

FIG. 1 illustrates a block diagram of an electrical device according to one embodiment of this invention. The electrical device adjusts size of at least one display icon displayed on the display unit of the electrical device according to physical information of a user.

The electrical device 100 includes a display unit 110 and a processing unit 120, which are electrically connected with each other. The processing unit 120 includes an information receiving module 121, a weight obtaining module 122 and a size adjusting module 123. The information receiving module 121 receives physical information of a user. Wherein, the physical information of the user may include the user's age, the user's visual acuity, the user's finger size or any other physical information of the user. Besides, the electrical device 100 may further include a user interface 140, which is electrically connected with the processing unit 120. Hence, users can input their physical information through the user interface 140. The user interface 140 may be a keyboard, buttons, a Graphical User Interface (GUI), a touch screen or any other user interface. Therefore, in one embodiment of this invention, the electrical device 100 may display a physical information input page on the display unit 110 for users to input their physical information through the user interface 140.

The weight obtaining module 122 obtains a display size weight according to the physical information of the user. In one embodiment, the weight obtaining module 122 may obtain the physical information of the user by checking a table about physical information/display size weight. In another embodiment, the weight obtaining module 122 may utilize a mathematical formula to calculate the display size weight according to the physical information of the user. In other embodiments, the display size weight can be obtained through other methods, which should not be limited in this disclosure.

The size adjusting module 123 adjusts size of at least one display icon displayed on the display unit 110 according to the display size weight. Wherein, the least one display icon may include a character, a photo or any other element for an icon. Therefore, the size of the display icon may be displayed to fit different users' physical condition without complicated setting steps.

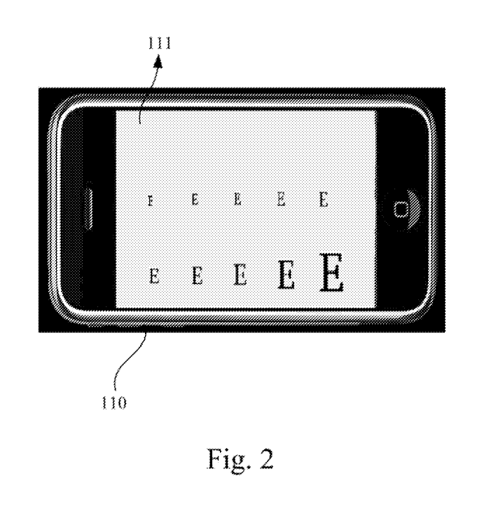

In one embodiment, the physical information may include visual acuity information of the user to be a factor for obtaining the display size weight. Wherein, users can input their visual acuity information through the user interface 140. Besides, the electrical device may provide a visual acuity test to obtain the visual acuity information of the user. FIG. 2 is an embodiment of a display appearance of the display unit 110 in FIG. 1. Refer to both FIG. 1 and FIG. 2. The processing unit 120 may further include a visual acuity testing module 124 and a visual acuity calculating module 125. The visual acuity testing module 124 makes the display unit 110 display a visual acuity test 111 for users to take. Users can take the visual acuity test 111 and generate an input signal for the visual acuity test 111 through the user interface 140. The visual acuity calculating module receives the input signal for the visual acuity test 111 and calculates the visual acuity information of the user according to the input signal. Wherein, the visual acuity information of the user is taken as the physical information of the user to be a factor to adjust size of the display icon. The better the user's visual acuity is, the smaller the size of the at least one display icon may be adjusted to by setting the value of the display size weight; the worse the user's visual acuity is, the bigger the size of the at least one display icon may be adjusted to by setting the value of the display size weight. Therefore, users with bad visual acuity can see the display icon displayed on the display unit 110 easily without complicated setting steps.

In another embodiment of this invention, the physical information may include the user's age to be a factor for obtaining the display size weight. Wherein, users can input their age information through the user interface 140. In addition, the electrical device 100 may identify the age information of the user through the user's image. Therefore, the electrical device 100 may include an image sensor 150, which is electrically connected with the processing unit 120, and the processing unit 120 may further include an image receiving module 126 and an age identifying module 127. The image receiving module 126 receives the user's image, which is sensed by the image sensor 150. The age identifying module 127 identifies age information of the user according to the user's image. Besides, the age information of the user may be identified according to the finger size of the user. Therefore, the display unit 110 may be a touch screen, and the processing unit 120 may further include a finger size calculating module 128 and an age converting module 129. The finger size calculating module 128 receives a measure of a touched area of the touch screen (the display unit 110), and calculates the finger size of the user according to the measure of the touched area. The age converting module 129 converts the finger size of the user into age information of the user. Wherein, the bigger the finger size of the user is, the older the user may be identified; the smaller the finger size of the user is, the younger the user may be identified. In other embodiments, the electrical device 100 can obtain the age information of the user through other methods, which should not be limited in this disclosure.

Then, the weight obtaining module 122 may take the age information of the user as the physical information of the user to be a factor to obtain the display size weight. Wherein, the younger the user is, the smaller the size of the at least one display icon may be adjusted to by setting the value of the display size weight; the older the user is, the bigger the size of the at least one display icon may be adjusted to by setting the value of the display size weight. Therefore, the elders can see the display icon displayed on the display unit 110 easily without complicated setting steps.

In addition, if the display unit 110 is a touch screen, users may not touch the display icon, size of which is smaller than users' finger, easily. Hence, the weight obtaining module 122 may obtain the display size weight, which makes the at least one display icon displayed on the touch screen (display unit 110) bigger than the finger size of the user. Therefore, the display icon on the display unit 110 can be touched for operation easily. In other embodiments, the weight obtaining module 122 can obtain the display size weight according to the physical information with several parameters utilizing a mathematical formula or by checking a corresponding table, which should not be limited in this disclosure.

In addition, the display icon should be adjusted to the size, which can be displayed by the display unit 110. Hence, the processing unit 110 may further include a size obtaining module 130. The size obtaining module 130 obtains a maximal size threshold and a minimal size threshold for the at least one display icon to be displayed on the display unit 110. The size adjusting module 123 adjusts the size of the at least one display icon displayed on the display unit 110 between the maximal size threshold and the minimal size threshold. Therefore, the display unit 110 of the electrical device 100 can display the adjusted display icon.

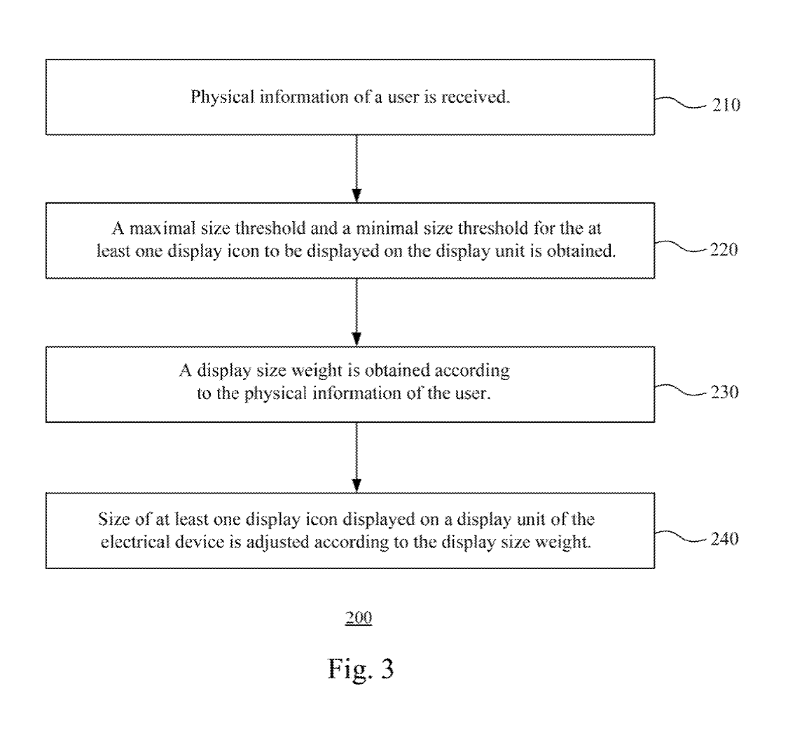

FIG. 3 is a flow diagram of a display method for an electrical device according to another embodiment of this invention. In the display method, size of at least one display icon displayed on the display unit of the electrical device is adjusted according to physical information of a user. The display method may take the form of a computer program product stored on a computer-readable storage medium having computer-readable instructions embodied in the medium. Any suitable storage medium may be used including non-volatile memory such as read only memory (ROM), programmable read only memory (PROM), erasable programmable read only memory (EPROM), and electrically erasable programmable read only memory (EEPROM) devices; volatile memory such as SRAM, DRAM, and DDR-RAM; optical storage devices such as CD-ROMs and DVD-ROMs; and magnetic storage devices such as hard disk drives and floppy disk drives. The display method 200 includes the following steps:

In step 210, physical information of a user is received. Wherein, the physical information of the user may include the user's age, the user's visual acuity, the user's finger size or any other physical information of the user. In one embodiment, the physical information of the user can be received through a user interface, such as a keyboard, buttons, a GUI, a touch screen or any other user interface. In other embodiments, the physical information can be received through other methods, which should not be limited in this disclosure.

In step 230, a display size weight is obtained according to the physical information of the user. In one embodiment, the physical information of the user can be obtained by checking a table about physical information/display size weight. In another embodiment, a mathematical formula may be utilized for calculating the display size weight according to the physical information of the user. In other embodiments, the display size weight can be obtained through other methods, which should not be limited in this disclosure.

In step 240, size of at least one display icon displayed on a display unit of the electrical device is adjusted according to the display size weight. Wherein, the least one display icon may include a character, a photo or any other element for an icon. Therefore, the size of the display icon may be displayed to fit different users' physical condition.

In one embodiment, the physical information may include visual acuity information of the user to be a factor for obtaining the display size weight (step 230). Wherein, users can input their visual acuity information through the user interface. Besides, a visual acuity test may be provided to obtain the visual acuity information of the user. Refer to both FIG. 2 and FIG. 3. Hence, in the display method 200; a visual acuity test 111 may be displayed on the display unit 110 for users to take. Users can take the visual acuity test 111 and generate an input signal for the visual acuity test 111 through the user interface. The input signal for the visual acuity test 111 may be received and the visual acuity information of the user may be calculated according to the input signal. Wherein, the visual acuity information of the user is taken as the physical information of the user to be a factor to adjust size of the display icon (step 240). The better the user's visual acuity is, the smaller the size of the at least one display icon may be adjusted to by setting the value of the display size weight; the worse the user's visual acuity is, the bigger the size of the at least one display icon may be adjusted to by setting the value of the display size weight. Therefore, users with bad visual acuity can see the display icon displayed on the display unit 110 easily without complicated setting steps.

In another embodiment of this invention, the physical information may include the user's age to be a factor for obtaining the display size weight (step 230). Wherein, users can input their age information through the user interface. In addition, the age information of the user may be identified according to the user's image. Therefore, in the display method 200, the user's image may be received. Then, age information of the user may be identified according to the user's image. Besides, the age information of the user may be identified according to the finger size of the user. Hence, if the display unit is a touch screen, a measure of a touched area of the touch screen may be received and the finger size of the user may be calculated according to the measure of the touched area. Then, the finger size of the user may be converted into age information of the user. Wherein, the bigger the finger size of the user is, the older the user may be identified; the smaller the finger size of the user is, the younger the user may be identified. In other embodiments, the display method 200 can obtain the age information of the user through other methods, which should not be limited in this disclosure.

Then, the display size weight can be obtained according to the age information of the user (step 230), and the size of the display icon can be adjusted according to the display size weight (step 240). Wherein, the younger the user is, the smaller the size of the at least one display icon may be adjusted to by setting the value of the display size weight; the older the user is, the bigger the size of the at least one display icon may be adjusted to by setting the value of the display size weight. Therefore, the elders can see the display icon displayed on the display unit easily without complicated setting steps.

In addition, if the display unit is a touch screen, users may not touch the display icon, size of which is smaller than users' finger, easily. Hence, the obtained display size weight should set to the values which makes the at least one display icon displayed on the touch screen bigger than the finger size of the user. Therefore, the display icon on the display unit can be touched for operation easily. Besides, in other embodiments, the display size weight can be obtained according to the physical information with several parameters utilizing a mathematical formula or by checking a corresponding table, which should not be limited in this disclosure.

In addition, the display icon should be adjusted to the size, which can be displayed by the display unit. Hence, in step 220, a maximal size threshold and a minimal size threshold for the at least one display icon to be displayed on the display unit 110 may be obtained. Then, in step 240, the size of the at least one display icon displayed on the display unit may be adjusted between the maximal size threshold and the minimal size threshold. Therefore, the display unit can display the adjusted display icon.

Above all, the size of the display icon may be displayed to fit different users' physical condition without complicated setting steps. Besides, the elders or the users with bad visual acuity can see the display icon displayed on the display unit easily without complicated setting steps.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. An electrical device comprising:
a display unit; and
a processing unit, electrically connected with the display unit, wherein the processing unit comprises:
an information receiving module for receiving physical information of a user;
a weight obtaining module for obtaining a display size weight according to the physical information of the user; and
a size adjusting module for adjusting size of at least one display icon displayed on the display unit according to the display size weight.

2. The electrical device of claim 1, wherein the electrical device further comprises a user interface, the user interface is electrically connected with the processing, the processing unit further comprises:
a visual acuity testing module for making the display unit display a visual acuity test; and
a visual acuity calculating module for calculating visual acuity information of the user according to an input signal for the visual acuity test, which is received through the user interface, wherein the visual acuity information of the user is taken as the physical information of the user.

3. The electrical device of claim 1, wherein the electrical device further comprises an image sensor, the image sensor is electrically connected with the processing unit, the processing unit further comprises:
an image receiving module for receiving the user's image, which is sensed by the image sensor; and
an age identifying module for identifying age information of the user according to the user's image, wherein the age information of the user is taken as the physical information of the user.

4. The electrical device of claim 1, wherein the display unit is a touch screen, the processing unit further comprises:
a finger size calculating module for receiving a measure of a touched area of the touch screen, and calculating a finger size of the user according to the measure of the touched area,
wherein the finger size of the user is taken as the physical information of the user,
wherein the weight obtaining module obtains the display size weight, which makes the at least one display icon displayed on the touch screen bigger than the finger size of the user.

5. The electrical device of claim 1, wherein the display unit is a touch screen, the processing unit further comprises:
a finger size calculating module for receiving a measure of a touched area of the touch screen, and calculating a finger size of the user according to the measure of the touched area; and
an age converting module for converting the finger size of the user into age information of the user, wherein the age information of the user is taken as the physical information of the user.

6. The electrical device of claim 1 further comprising:
a user interface, electrically connected with the processing unit, wherein the physical information of the user is received through the user interface.

7. The electrical device of claim 1, wherein the processing unit further comprises:
a size obtaining module for obtaining a maximal size threshold and a minimal size threshold for the at least one display icon to be displayed on the display unit, and adjusting the size of the at least one display icon displayed on the display unit between the maximal size threshold and the minimal size threshold according to the display size weight.

8. A display method for an electrical device comprising:
receiving physical information of a user;
obtaining a display size weight according to the physical information of the user; and
adjusting size of at least one display icon displayed on a display unit of the electrical device according to the display size weight.

9. The display method of claim 8 further comprising:
displaying a visual acuity test on the display unit;
receiving an input signal for the visual acuity test; and
calculating visual acuity information of the user according to the input signal, wherein the visual acuity information of the user is taken as the physical information of the user.

10. The display method of claim 8 further comprising:
receiving the user's image; and
identifying age information of the user according to the user's image, wherein the age information of the user is taken as the physical information of the user.

11. The display method of claim 8, wherein the display unit is a touch screen, the display method further comprises:
receiving a measure of a touched area of the touch screen; and
calculating a finger size of the user according to the measure of the touched area,
wherein the finger size of the user is taken as the physical information of the user,
wherein the display size weight is obtained according to the finger size of the user, such that the at least one display icon displayed on the touch screen is bigger than the finger size of the user.

12. The display method of claim 8, wherein the display unit is a touch screen, the display method further comprises:
receiving a measure of a touched area of the touch screen;
calculating a finger size of the user according to the measure of the touched area; and
converting the finger size of the user into age information of the user, wherein the age information of the user is taken as the physical information of the user.

13. The display method of claim 8 further comprising:
obtaining a maximal size threshold and a minimal size threshold for the at least one display icon to be displayed on the display unit,
wherein adjusting the size of the at least one display icon displayed on the display unit comprises:
adjusting the size of the at least one display icon displayed on the display unit to fit the size between the maximal size threshold and the minimal size threshold.

14. The display method of claim 8, wherein the physical information of the user comprises a plurality of physical parameters of the user, and the display method further comprises:
obtaining the display size weight utilizing a mathematical formula or by checking a table according to the physical parameters of the user.

15. An electrical device readable storage medium with a computer program to execute a display method for an electrical device, wherein the display method comprises:
receiving physical information of a user;
obtaining a display size weight according to the physical information of the user; and
adjusting size of at least one display icon displayed on a display unit of the electrical device according to the display size weight.

16. The electrical device readable storage medium of claim 15, wherein the display method further comprises:
displaying a visual acuity test on the display unit;
receiving an input signal for the visual acuity test; and
calculating visual acuity information of the user according to the input signal, wherein the visual acuity information of the user is taken as the physical information of the user.

17. The electrical device readable storage medium of claim 15, wherein the display method further comprises:
receiving the user's image; and
identifying age information of the user according to the user's image, wherein the age information of the user is taken as the physical information of the user.

18. The electrical device readable storage medium of claim 15, wherein the display unit is a touch screen, the display method further comprises:
receiving a measure of a touched area of the touch screen; and
calculating a finger size of the user according to the measure of the touched area,
wherein the finger size of the user is taken as the physical information of the user,
wherein the display size weight is obtained according to the finger size of the user, such that the at least one display icon displayed on the touch screen is bigger than the finger size of the user.

19. The electrical device readable storage medium of claim 15, wherein the display unit is a touch screen, the display method further comprises:
receiving a measure of a touched area of the touch screen;
calculating a finger size of the user according to the measure of the touched area; and
converting the finger size of the user into age information of the user, wherein the age information of the user is taken as the physical information of the user.

20. The electrical device readable storage medium of claim 15, wherein the display method further comprises:
obtaining a maximal size threshold and a minimal size threshold for the at least one display icon to be displayed on the display unit,
wherein adjusting the size of the at least one display icon displayed on the display unit comprises:
adjusting the size of the at least one display icon displayed on the display unit to fit the size between the maximal size threshold and the minimal size threshold.
